(19) United States
(12) Patent Application Publication
(10) Pub. No.: US 2022/0192781 A1
(43) Pub. Date: Jun. 23, 2022

(54) Apparatus of Medical Endoscope Using Mipi Serializer/Deserializer

(71) Applicant: YUAN High-Tech Development Co., Ltd., Taipei (TW)

(72) Inventor: Shih-Huai Yang, Taipei (TW)

(21) Appl. No.: 17/224,257

(22) Filed: Apr. 7, 2021

(30) Foreign Application Priority Data
Dec. 18, 2020 (TW) 109216801

(51) Int. Cl. A61B 90/70; A61B 90/50; A61B 1/05; A61B 1/00

(52) U.S. Cl. CPC A61B 90/70; A61B 90/50; A61B 2090/701; A61B 1/00018; A61B 1/00009; A61B 1/05

(57) ABSTRACT

An apparatus of serializer/deserializer is provided. The apparatus comprises a holding unit and a capture unit. The holding unit connects to the capture unit through a coaxial cable, and comprises a disposable image-capture module, a synthetic-image module (L/R montage), a Mipi serializer, a Mipi deserializer, a Mipi capture card, and a capture device. Thus, the apparatus keeps the most expensive ISP and capture device at a back end; and puts the disposable image-capture module, the synthetic-image module, and the Mipi serializer at a front end. After use, it is only necessary to discard the disposable image-capture module, which extends into human body at the front end. The remaining parts are reused. Thus, cost is effectively reduced and the shortcoming of high cost of the use of modern disposable endoscope is solved.

### TECHNICAL FIELD OF THE INVENTION

The present invention relates to an apparatus of medical endoscope using Mipi serializer/deserializer; more particularly, to discarding a disposable image-capture module only with remaining parts reused, where cost is effectively reduced and the shortcoming of high cost of the use of modern disposable endoscope is solved.

### DESCRIPTION OF THE RELATED ARTS

With the advancement of image display technology, medical practices involving the use of endoscopes are becoming more and more widespread. A current medical endoscope device mostly comprises a lens, a light-emitting device, and a hollow tube. After connecting to an external monitor, the image inside a target can be seen.

The medical endoscope is in principle reusable. The host of endoscope along with its mirrors is very expensive. Therefore, after each use, manpower and time must be consumed for cleaning and disinfecting to ensure no infection for next use. However, medical personnel have faced a shortage in recent years. If the post-processing after using the endoscope could be simplified, more convenience would be brought to medical behaviors.

In the existing technical field of medical equipment for solving the problems of insufficient manpower and insufficient cleaning and disinfection, technical concept of discarding is accepted for endoscope. This kind of endoscope has a long tube for inserting into an opening of a body. The inserting tube of the endoscope usually comprises a system of optical fiber and lens for carrying the assembled visual information out of a patient's body; and a light source for illuminating the area to be seen. In the design of this disposable endoscope, the following parts are combined into a single integrated entity: a detector (including a lighting, a photographer, etc.), an image signal processor (ISP), an interface of universal serial bus (USB), etc., where all expensive parts of the disposable endoscope are designed at the front end of the tube and the entire tube with the inserted parts is discarded after use. Yet, this method is expensive due to the high cost of imaging optics. The throwing away after use is not only very wasteful but also inconsistent with economic benefits. Hence, the prior arts do not fulfill all users' requests on actual use.

### SUMMARY OF THE INVENTION

The main purpose of the present invention is to set the most expensive ISP and capture device at a back end with a disposable image-capture module, a synthetic-image module, and a Mipi serializer at a front end; and, after use, only discard the disposable image-capture module, which extends into human body at the front end, but keep the other parts reusable, where cost is thus effectively reduced to solve the shortcoming of high cost of the use of modern disposable endoscope.

To achieve the above purposes, the present invention is an apparatus of medical endoscope using Mipi serializer/deserializer, comprising a holding unit and a capture unit, where the holding unit and the capture unit are connected through a coaxial cable, and comprises a disposable image-capture module, a synthetic-image module, a Mipi serializer, a Mipi deserializer, a Mipi capture card, and a capture device; the disposable image-capture module is set at a first end of the holding unit; the disposable image-capture module comprises a left image sensor and a right image sensor; the left image sensor and the right image sensor capture a set of left-image signals and a set of right-image signals, respectively; the set of left-image signals and the set of right-image signals are converted for conforming to a Mipi single-channel (Mipi-1-lane) interface; the synthetic-image module is set in the holding unit and connects to the disposable image-capture module; the synthetic-image module receives the set of left-image signals and the set of right-image signals to form a set of synthesized image signals containing all of the left-image signals and the right-image signals; the set of synthesized image signals are converted for conforming to a Mipi dual-channel (Mipi-2-lane) interface; the Mipi serializer is set in the holding unit and connects to the synthetic-image module; the Mipi serializer Mipi-serializes the set of synthesized image signals for outputting a flow of Mipi-serialized image data to the coaxial cable; the Mipi deserializer is set in the capture unit and connects to the Mipi serializer through the coaxial cable to Mipi-deserialize the flow of Mipi-serialized image data to be restored and conformed to the Mipi-2-lane interface for outputting the set of synthesized image signals; the Mipi capture card is set in the capture unit and connects to the Mipi deserializer for receiving the set of synthesized image signals to be converted into a set of parallel signals; and the capture device is set in the capture unit and connects to the Mipi serializer to receive the set of parallel signals for being converted into a set of video-format data. Accordingly, a novel apparatus of medical endoscope using Mipi serializer/deserializer is obtained.

### BRIEF DESCRIPTION OF THE DRAWINGS

The present invention will be better understood from the following detailed description of the preferred embodiment according to the present invention, taken in conjunction with the accompanying drawings, in which

FIG.1 is the view showing the structure of the preferred embodiment according to the present invention; and

FIG.2 is the view showing the detail of the structure.

### DESCRIPTION OF THE PREFERRED EMBODIMENT

The following description of the preferred embodiment is provided to understand the features and the structures of the present invention.

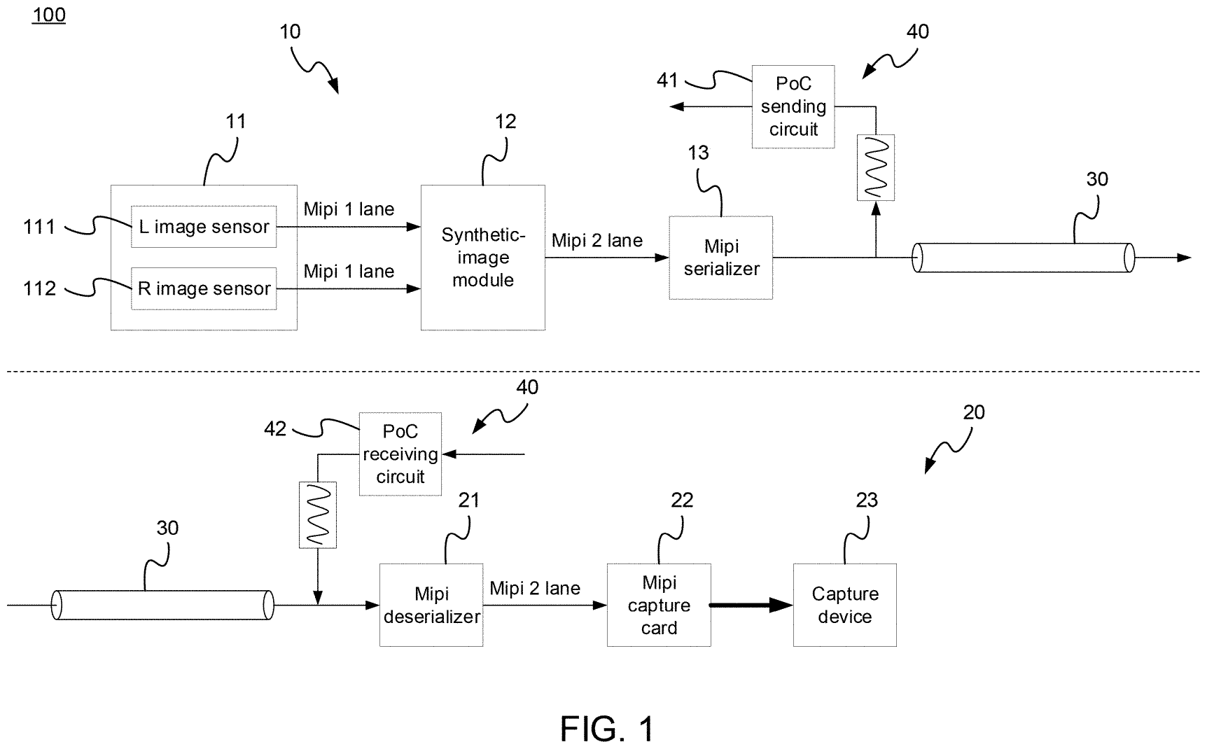

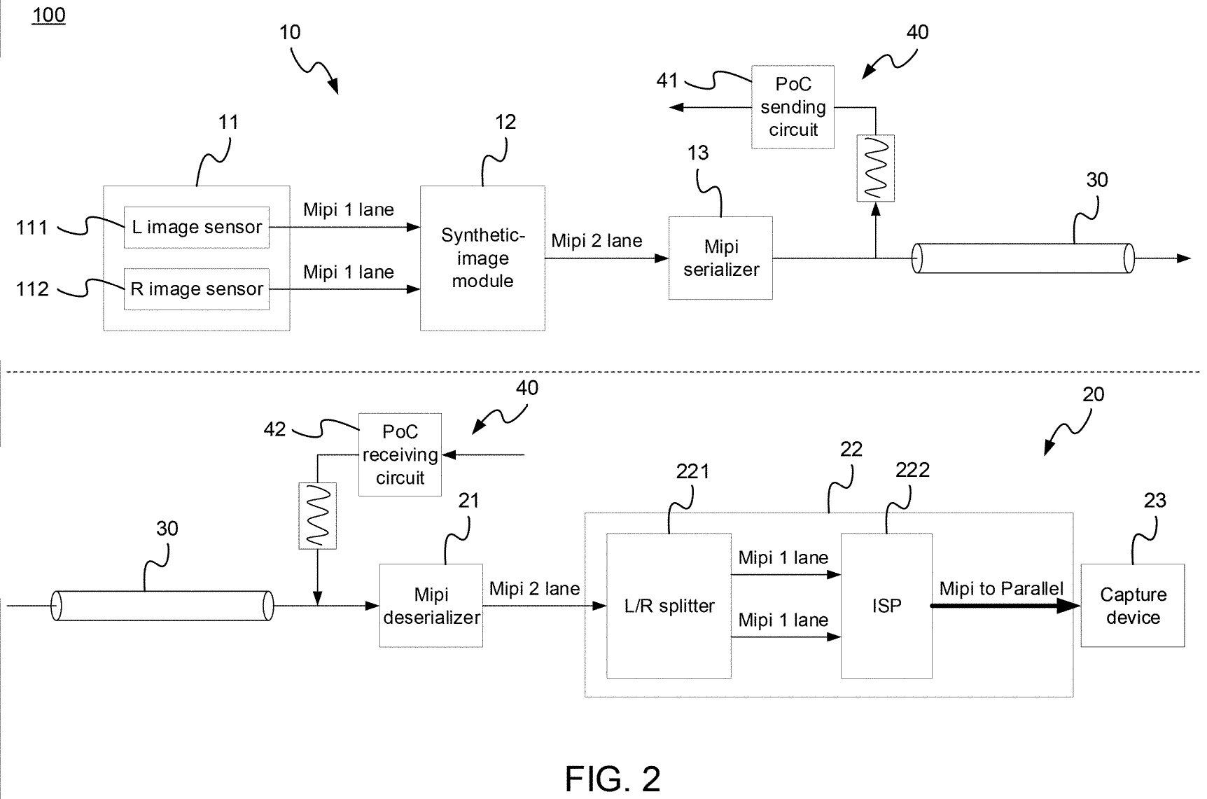

Please refer to FIG.1 and FIG.2, which are a view showing the structure of a preferred embodiment according to the present invention; and a view showing the detail of the structure. As shown in the figures, the present invention is an apparatus of medical endoscope using Mipi serializer/deserializer 100, comprising a holding unit 10 and a capture unit 20. The holding unit 10 and the capture unit 20 are connected through a coaxial cable 30, and comprise a disposable image-capture module 11, a synthetic-image module 12, a Mipi serializer 13, a Mipi deserializer 21, a Mipi capture card 22, and a capture device 23.

The disposable image-capture module 11 is set at a front end of the holding unit, and comprises a left image sensor 111 and a right image sensor 112. Therein, the front end of the holding unit 10 is a guide tube, which can extend into a body and has a length of at least 30 centimeters.

The synthetic-image module 12 is set in the holding unit 10 and connects to the disposable image-capture module 11.

The Mipi serializer 13 is set in the holding unit 10 and connects to the synthetic-image module 12.

The Mipi deserializer 21 is set in the capture unit 20 and connects to the Mipi serializer 13 through the coaxial cable 30, where the coaxial cable 30 has a length of 2-10 meters.

The Mipi capture card 22 is set in the capture unit 20 and connects to the Mipi deserializer 21.

The capture device 23 is set in the capture unit 20 and connects to the Mipi serializer 22.

The apparatus of medical endoscope using Mipi serializer/deserializer 100 further comprises a power-over-cable (PoC) module 40, where the PoC module 40 comprises a PoC sending circuit 41 set between the coaxial cable 30 and the Mipi deserializer 21; and a PoC receiving circuit 42 set between the Mipi serializer 13 and the coaxial cable 30. Thus, a novel apparatus of medical endoscope using Mipi serializer/deserializer 100 is obtained.

On using the present invention, the disposable image-capture module 11 at the front end of the holding unit 10 extends into the body to use the left image sensor 111 to capture a set of left-image signals and the right image sensor 112 to capture a set of right-image signals. The set of left-image signals (L signals) and the set of right-image signals (R signals) are converted to be conformed to a Mipi single-channel (Mipi-1-lane) interface.

The synthetic-image module 12 receives the L signals and the R signals to form a set of synthesized image signals (L+R signals) containing all of the L signals and the R signals. After the set of synthesized image signals are converted to be conformed to a Mipi dual-channel (Mipi-2-lane) interface, the Mipi serializer 13 Mipi-serializes the set of synthesized image signals to output a flow of Mipi-serialized image data to the coaxial cable 30.

In addition to data transmission, the apparatus of medical endoscope using Mipi serializer/deserializer 100 also uses a coaxial-cable power supplier 40 to supply power in a reverse direction. As shown in FIG.1, the PoC sending circuit 41 is configured to send power over the coaxial cable 30; and the PoC receiving circuit 42 is configured to receive power from the coaxial cable 30 for supplying power to the disposable image-capture module 11 at the front end of the holding unit 10.

The Mipi deserializer 21 receives the flow of Mipi-serialized image data through the coaxial cable 30 to Mipi-deserialize the flow of Mipi-serialized image data to be restored and conformed to the Mipi-2-lane interface for outputting the set of synthesized image signals to the Mipi capture card 22. In a state-of-use as shown in FIG.2, the Mipi capture card 22 has an L/R splitter 221, where the set of synthesized image signals is received to be split into two sets of image signals conformed to the Mipi-1-lane interface. Then, the two sets of image signals conformed to the Mipi-1-lane interface are outputted to an image signal processor (ISP) to be converted into two sets of image signals conformed to the Mipi-2-lane interface. The two sets of image signals conformed to the Mipi-2-lane interface are processed through a Mipi-to-parallel data conversion to obtain parallel signals compatible to the capture device 23. At last, the capture device 23 converts the parallel signals into video-format data.

Hence, the apparatus keeps the most expensive ISP and capture device at the back end; and puts the disposable image-capture module, the synthetic-image module, and the Mipi serializer at the front end. After use, it is only necessary to discard the disposable image-capture module whose front end extends into human body. The remaining parts are reused. Thus, cost is effectively reduced to solve the shortcoming of high cost of the use of modern disposable endoscope.

To sum up, the present invention is an apparatus of medical endoscope using Mipi serializer/deserializer, where the most expensive ISP and capture device are kept at a back endand a disposable image-capture module, a synthetic-image module, and a Mipi serializer are put at a front end; after use, it is only necessary to discard the disposable image-capture module, which extends into human body at the front end, but the remaining parts are reused; and cost is thus effectively reduced to solve the shortcoming of high cost of the use of modern disposable endoscope.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. An apparatus of medical endoscope using Mipi serializer/deserializer,
comprising a holding unit and a capture unit,
wherein said holding unit and said capture unit are connected through a coaxial cable, and comprises a disposable image-capture module,
wherein said disposable image-capture module is deposed at a first end of said holding unit; said disposable image-capture module comprises a left image sensor and a right image sensor; said left image sensor and said right image sensor capture a set of left-image signals and a set of right-image signals, respectively; and said set of left-image signals and said set of right-image signals are converted to be conformed to a Mipi single-channel (Mipi-1-lane) interface;
a synthetic-image module,
wherein said synthetic-image module is deposed in said holding unit and connects to said disposable image-capture module; said synthetic-image module receives said set of left-image signals and said set of right-image signals to obtain a set of synthesized image signals containing all of said left-image signals and said right-image signals; and said set of synthesized image signals are converted to be conformed to a Mipi dual-channel (Mipi-2-lane) interface;
a Mipi serializer,
wherein said Mipi serializer is deposed in said holding unit and connects to said synthetic-image module; said Mipi serializer Mipi-serializes said set of synthesized image signals to output a flow of Mipi-serialized image data to said coaxial cable;
a Mipi deserializer,
wherein said Mipi deserializer is deposed in said capture unit and connects to said Mipi serializer through said coaxial cable to Mipi-deserialize said flow of Mipi-serialized image data to be restored and conformed to said Mipi-2-lane interface to output said set of synthesized image signals;
a Mipi capture card,
wherein said Mipi capture card is deposed in said capture unit and connects to said Mipi deserializer to receive said set of synthesized image signals to be converted into a set of parallel signals; and
a capture device,
wherein said capture device is deposed in said capture unit and connects to said Mipi serializer to receive said set of parallel signals to be converted into a set of video-format data.

2. The apparatus according to claim 1,
wherein said first end of said holding unit is a guide tube; said guide tube extends into a body; and has a length of at least 30 centimeters.

3. The apparatus according to claim 1,
wherein the apparatus further comprises a power-over-cable (PoC) module and said PoC module comprises
a PoC sending circuit, wherein said PoC sending circuit is deposed between said coaxial cable and said Mipi deserializer to be configured to send power over said coaxial cable; and
a PoC receiving circuit, wherein said PoC receiving circuit is deposed between said Mipi serializer and said coaxial cable and configured to receive power from said coaxial cable to supply power to said disposable image-capture module at said first end of said holding unit.

4. The apparatus according to claim 1,
wherein said Mipi capture card has an L/R splitter to receive said set of synthesized image signals to be split into two sets of image signals conformed to said Mipi-1-lane interface; then, said two sets of image signals conformed to said Mipi-1-lane interface are outputted to an image signal processor (ISP) to be converted into two sets of image signals conformed to said Mipi-2-lane interface; and said two sets of image signals conformed to said Mipi-2-lane interface are processed through a Mipi-to-parallel data conversion to obtain parallel signals compatible to said capture device.

5. The apparatus according to claim 1,
wherein said coaxial cable has a length of 2˜10 meters.
